(19) United States
(12) Patent Application Publication
(10) Pub. No.: US 2025/0022125 A1
(43) Pub. Date: Jan. 16, 2025

(54) IMAGE PROCESSING METHOD AND COMPUTING DEVICE

(71) Applicant: Our United Corporation, Shaanxi (CN)

(72) Inventors: Hao YAN, Shaanxi (CN); Dalin LIU, Shaanxi (CN); Minfei LU, Shaanxi (CN); Zhongya WANG, Shaanxi (CN); Jinsheng LI, Shaanxi (CN)

(21) Appl. No.: 18/381,028

(22) Filed: Oct. 17, 2023

(30) Foreign Application Priority Data
Jul. 10, 2023 (CN) 202310840556.0

(51) Int. Cl. G06T 7/00; G06T 7/30; G06V 10/46; G16H 20/40

(52) U.S. Cl. CPC G06T 7/0012; G06T 7/30; G06V 10/46; G16H 20/40; G06T 2207/10081; G06T 2207/30096

(57) ABSTRACT

An image processing method includes: obtaining a guidance image, the guidance image being an image generated based on an image-guided radiation therapy system; and performing image processing on the guidance image to generate a target image, the target image being a computed tomography simulation image corresponding to the guidance image.

### CROSS-REFERENCE TO RELATED APPLICATION

This application claims priority to Chinese Patent Application No. 202310840556.0, filed on Jul. 10, 2023, which is incorporated herein by reference in its entirety.

### TECHNICAL FIELD

The present disclosure relates to the field of radiation therapy technologies, and in particular, to an image processing method and a computing device.

### BACKGROUND

Radiation therapy is one of the commonly treatments used for cancer. At present, radiation therapy systems are generally equipped with image guidance devices, which use image guidance technology to assist in determining the tumor target volume and at the same time help dose implementation, thus improving the accuracy and efficiency of radiation therapy.

Image guidance technology generally uses cone beam computed tomography (CBCT) images to guide human body positioning during radiation therapy. CBCT imaging can achieve fast imaging and has the characteristics of small size, low cost, and low radiation dose to the patient. Therefore, it is widely used for positioning correction and image reference during fractionated radiation therapy, i.e., for performing registration on planned CT images and CBCT images of radiation therapy.

### SUMMARY

In a first aspect, an image processing method is provided. The image processing method includes: obtaining a guidance image, the guidance image being an image generated based on an image-guided radiation therapy system; and performing image processing on the guidance image to generate a target image, the target image being a computed tomography (CT) simulation image corresponding to the guidance image.

In a second aspect, a computing device is provided. The computing device includes a processor and a memory. The processor is coupled to the memory. The memory is used to store one or more programs. The one or more programs include computer program instructions that, when executed by the processor, cause the computing device to perform the image processing method as described in the first aspect.

In a third aspect, a non-transitory computer-readable storage medium is provided. The non-transitory computer-readable storage medium has stored computer program instructions that, when run on a computer, cause the computer to perform the computing device to perform the image processing method as described in the first aspect.

### BRIEF DESCRIPTION OF THE DRAWINGS

In order to describe technical solutions in the present disclosure more clearly, the accompanying drawings to be used in some embodiments of the present disclosure will be introduced briefly. However, the accompanying drawings to be described below are merely drawings of some embodiments of the present disclosure, and a person of ordinary skill in the art can obtain other drawings according to those drawings. In addition, the accompanying drawings in the following description may be regarded as schematic diagrams, but are not limitations on actual sizes of products, actual processes of methods and actual timings of signals involved in the embodiments of the present disclosure.

FIG. 1A is a schematic diagram of a radiation therapy system provided in some embodiments of the present disclosure;

FIG. 1B is a diagram showing an example of a tumor movement trajectory in a respiratory cycle provided in some embodiments of the present disclosure;

FIG. 2 is a schematic flowchart of an image processing method provided in some embodiments of the present disclosure;

FIG. 3 is a schematic flowchart of another image processing method provided in some embodiments of the present disclosure;

FIG. 4 is a schematic diagram of an image processing method provided in some embodiments of the present disclosure;

FIG. 5, FIG. 6, FIG. 7, FIG. 8, FIG. 9, FIG. 10 and FIG. 11 are flowcharts of an image processing method provided in some other embodiments of the present disclosure;

FIG. 12 is a schematic diagram of display images provided in some embodiments of the present disclosure;

FIG. 13 is a schematic diagram of prompt information provided in some embodiments of the present disclosure;

FIG. 14A is a schematic diagram of display images provided in some other embodiments of the present disclosure;

FIG. 14B is a schematic diagram of prompt information provided in some other embodiments of the present disclosure;

FIG. 15A is a schematic flowchart of yet another image processing method provided in some embodiments of the present disclosure;

FIG. 15B is a schematic flowchart of yet another image processing method provided in some embodiments of the present disclosure;

FIG. 16 is a schematic flowchart of yet another image processing method provided in some embodiments of the present disclosure;

FIG. 17 is a schematic flowchart of yet another image processing method provided in some embodiments of the present disclosure;

FIG. 18 is a schematic structural diagram of an image processing device provided in some embodiments of the present disclosure;

FIG. 19 is a schematic diagram of a computing device provided in some embodiments of the present disclosure; and

FIG. 20 is a partial schematic diagram of a computer program product provided in some embodiments of the present disclosure.

### DETAILED DESCRIPTION

The technical solutions in some embodiments of the present disclosure will be described clearly and completely below with reference to the accompanying drawings. However, the described embodiments are merely some but not all of embodiments of the present disclosure. All other embodiments obtained on the basis of the embodiments of the present disclosure by a person of ordinary skill in the art shall be included in the protection scope of the present disclosure.

Unless the context requires otherwise, throughout the description and claims, the term “comprise” and other forms thereof such as the third-person singular form “comprises” and the present participle form “comprising” are construed as an open and inclusive meaning, i.e., “included, but not limited to”. For example, a process, method, system, product or device that includes a series of steps or modules is not limited to the listed steps or modules, but optionally also includes other unlisted steps or modules, or optionally also includes other steps or modules that are inherent to such process, method, product, or device.

In the description of the specification, the term such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above term do not necessarily refer to the same embodiment(s) or example(s). In addition, specific features, structures, materials or characteristics may be included in any one or more embodiments or examples in any suitable manner. In addition, any embodiment or design described herein as the term such as “for example” or the like is not intended to be construed as preferred or advantageous over other embodiments or designs.

In the description of some embodiments, the terms “coupled” and “connected” and derivatives thereof may be used. For example, the term “connected” may be used in the description of some embodiments to indicate that two or more components are in direct physical or electrical contact with each other. As another example, the term “connected” may be used in the description of some embodiments to indicate that two or more components are in direct physical or electrical contact. However, the term “connected” or “communicatively connected” may also mean that two or more components are not in direct contact with each other, but still cooperate or interact with each other. The embodiments disclosed herein are not necessarily limited to the context herein.

Herein, the symbol “/” is used to indicate that the relationship between the related objects before and after is “or”. For example, the phrase “A/B” can be understood as A or B. The phrase “A and/or B” includes the following three combinations: only A, only B, and a combination of A and B.

As used herein, depending on the context, the term “if” is optionally construed as “when”, “in a case where”, “in response to determining” or “in response to detecting”. Similarly, depending on the context, the phrase “if it is determined” or “if [a stated condition or event] is detected” is optionally construed as “in a case where it is determined”, “in response to determining”, “in a case where [the stated condition or event] is detected”, or “in response to detecting [the stated condition or event]”.

The phrase “applicable to” or “configured to” as used herein indicates an open and inclusive expression, which does not exclude devices that are applicable to or configured to perform additional tasks or steps.

Hereinafter, terms such as “first” and “second” are only used for descriptive purposes, and are not to be construed as indicating or implying the relative importance or implicitly indicating the number of indicated technical features. Thus, features defined with “first” and “second” may explicitly or implicitly include one or more of the features. In addition, the terms “first” and “second” are used to distinguish different objects and are not used to describe a specific order of the objects.

In the description of the embodiments of the present disclosure, term “a plurality of” or “the plurality of” means two or more unless otherwise specified.

Firstly, application scenarios of some embodiments of the present disclosure will be introduced.

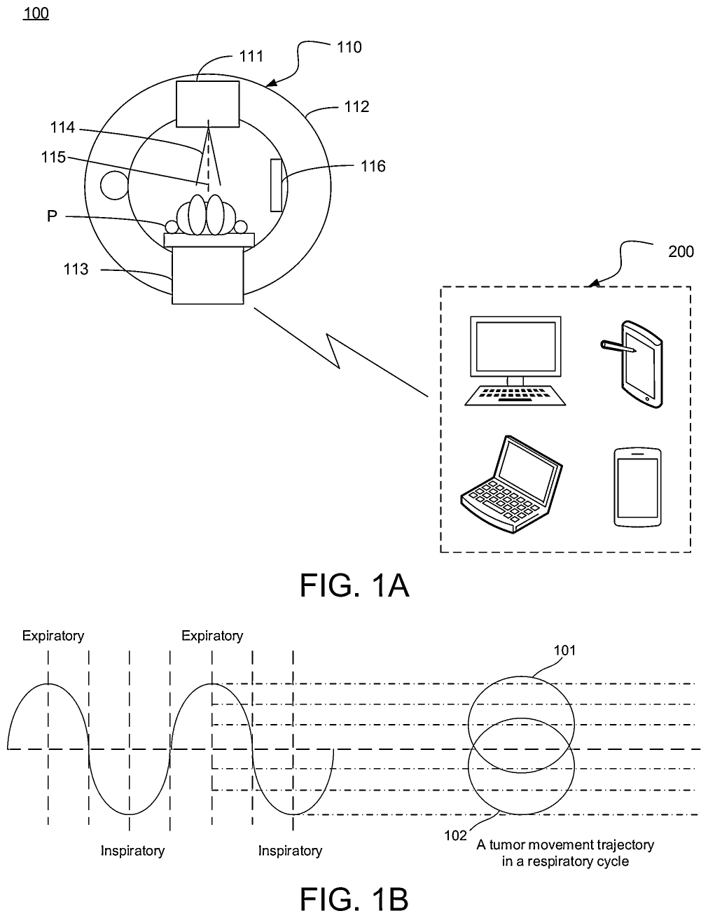

FIG. 1A is a schematic diagram of a radiation therapy system 100 (i.e., an image-guided radiation therapy system) provided in some embodiments of the present disclosure. The radiation therapy system 100 may include a radiation therapy device 110 and a data processing device 200. The radiation therapy device 110 may include a support device 113, a rotation frame 112, a radiation source 111 disposed on the rotation frame 112, and a group of image acquisition components.

The radiation source 111 is used to generate a beam 114 (an electron beam or X-ray) at a high energy level (e.g., megavolt (MV) level), so as to treat a target volume (including a tumor).

The support device 113 may be used to support a patient P, and may further move and position the patient P. For example, the support device 113 may be a three-dimensional, four-dimensional, five-dimensional or six-dimensional treatment bed or treatment chair.

For example, a group of image acquisition components may be disposed on the rotation frame 112. Each group of image acquisition components includes a tube 115 and a detector 116 arranged opposite each other, and is used for cone-beam computed tomography imaging.

The rotation frame 112 may be used to drive the tube 115 and the detector 116 to rotate. The rotation frame 112 may be a ring-shaped frame, a C-arm frame, a drum-shaped frame, etc. The embodiments of the present disclosure do not limit the structure of the rotation frame, which will be described based on the examples shown in the drawings.

In some embodiments, the tube 115 is a ray emission source, which may also be called a ray tube, or an imaging source. The tube 115 may be used to emit a cone imaging beam (e.g., X-rays at kilovolt (KV) level). The tube 115 may include a linear accelerator. The detector 116 may be a flat panel detector or a curved detector. The detector 116 is used to receive the imaging beam emitted by the tube 115. The detector 116 may generate a corresponding two-dimensional image or three-dimensional image according to the received imaging beam emitted by the tube 115. In some embodiments, the detector 116 may be, for example, a computed tomography (CT) device, a cone beam CT device, a positron emission tomography (PET) device, a volume CT device, or their combinations.

The data processing device 200 is communicatively connected to the detector 116. In some examples, the data processing device 200 is provided at a location far away from the radiation therapy device 110. For example, the data processing device 200 and the radiation therapy device 110 are located in different rooms, so as to protect the operator from radiation. The data processing device 200 may be a computer (or mobile phone, tablet computer, notebook computer, etc.) for controlling the radiation therapy system 100. The data processing device 200 may further receive a projection image from the detector 116 and perform processing on the projection image. The data processing device 200 may include an input device (e.g., a keyboard) for receiving input information, and may further include a display for displaying information before or during radiation therapy.

In some embodiments, the data processing device 200 is a computing device with a graphical user interface (GUI). The computing device may include one or more processors and a memory, and may further include one or more application programs.

It should be noted that the image-guided radiation therapy system shown in FIG. 1A is only an example to more clearly illustrate the technical solutions of the embodiments of the present disclosure, and does not constitute a limitation on the technical solutions provided by the embodiments of the present disclosure. Those of ordinary skill in the art will know that with the evolution of image-guided radiation therapy systems and the emergence of new task scenarios, the technical solutions provided by the embodiments of the present disclosure are also applicable to similar technical problems. The image processing method provided by some embodiments of the present disclosure can be applied in CBCT image processing scenarios.

At present, CBCT imaging is widely used for positioning correction and image reference during fractionated radiation therapy, i.e., for performing registration on planned CT images and CBCT images of radiation therapy. However, the image registration accuracy is low due to the poor quality of current CBCT images.

In addition, in the radiation therapy, respiratory movement is one of the very important factors in the accuracy of image radiation therapy. Since the lungs and abdomen move during every respiratory, there is a need to consider the impact of this movement on radiation in the radiation therapy. For example, as shown in FIG. 1B, in the expiratory cycle, the tumor is located in the area 101; and in the inspiratory cycle, the tumor is located in the area 102.

Currently, in order to ensure that radiation can be accurately irradiated to the tumor area, doctors usually use special equipment to track the respiratory movement of the lungs, such as respiratory gating and deep inspiration hold technologies. These methods allow patients to take deep breaths or hold their breath while undergoing radiation therapy, thereby reducing the disruption of respiratory movement during treatment. However, these methods are cumbersome, and the irradiation is performed only during a fixed time period, thus increasing the treatment time of the patients.

In order to solve the above problems, some embodiments of the present disclosure provide an image processing method. The method is used in a computing device (which may also be called a processing device, namely the above-mentioned data processing device 200). The processing device may obtain a guidance image, and the guidance image is an image generated based on an image-guided radiation therapy system (e.g., the above radiation therapy system 100). Then, the processing device may perform image processing on the guidance image to generate a target image, and the target image is a CT simulation image corresponding to the guidance image. Performing, by the processing device, the image processing on the guidance image includes: inputting the guidance image into an image conversion model trained based on deep learning; or performing deformable registration combined with forward and backward projection calculation on the guidance image. Since the CT image has the characteristic of clear image, the embodiments of the present disclosure obtain the CT simulation image through the guidance image, which may effectively solve the problem of poor CBCT image quality. Moreover, when the CT simulation image has high definition, the image registration accuracy may be effectively improved. In addition, by adopting the technical solutions provided in the embodiments of the present disclosure, there is no need to add special device and control the respiratory cycle, and thus the efficiency of treatment is improved.

The image processing method provided in the embodiments of the present disclosure is exemplarily described below with reference to the accompanying drawings.

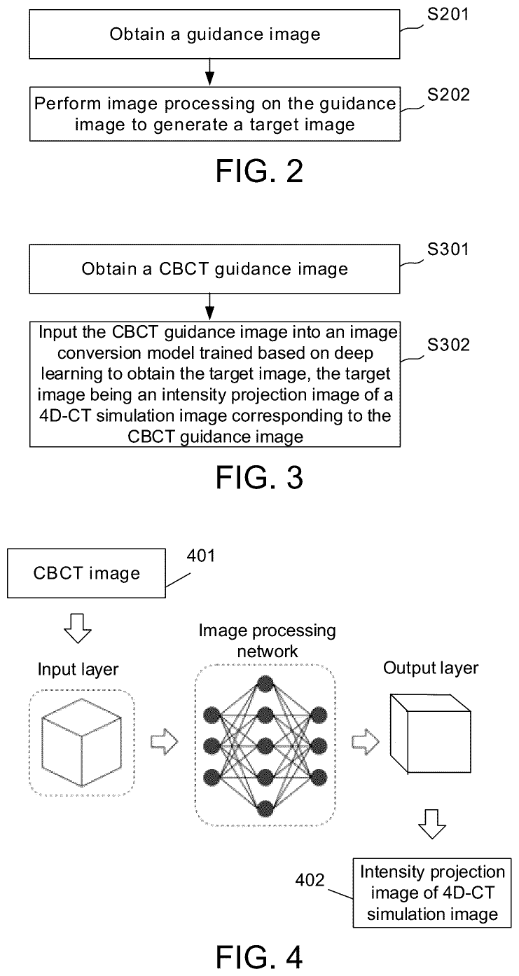

As shown in FIG. 2, the image processing method includes the following steps.

In S201, a guidance image is obtained.

The guidance image is the image generated based on the image-guided radiation therapy system (e.g., the above radiation therapy system 100 shown in FIG. 1A).

It should be noted that the guidance image is used to guide human body positioning during radiation therapy.

In a possible implementation, the guidance image is a CBCT guidance image or a four-dimensional-CBCT (4D)-CBCT guidance image. In the embodiments of the present disclosure, the 4D-CBCT guidance image is used to indicate the movement trajectory of the tumor during the respiratory cycle. The 4D-CBCT guidance image may include: CBCT images of different phases within a respiratory cycle. For example, if within a respiratory cycle, 1st to 2nd seconds are the inspiration cycle and 3rd to 4th seconds are the expiration cycle, then the 4D-CBCT guidance image may include: CBCT images in the inspiration cycle of the 1st to 2nd seconds, and CBCT images in the expiration cycle of the 3rd to 4th seconds.

In S202, image processing is performed on the guidance image to generate a target image. The target image is the CT simulation image corresponding to the guidance image. That is to say, in the embodiment of the present disclosure, the generated target image is the CT simulation image. It can be understood that the CT image has high definition, and by generating the CT simulation image through the guidance image, the poor image quality of the guidance image may be compensated.

In the embodiments of the present disclosure, performing the image processing on the guidance image may include: inputting the guidance image into an image conversion model trained based on deep learning; or performing deformable registration combined with forward and backward projection calculation on the guidance image.

That is to say, in the embodiments of the present disclosure, the processing device may input the guidance image into the trained image conversion model to obtain the target image. Alternatively, the processing device may perform the deformable registration combined with forward and backward projection calculation on the guidance image to obtain the target image.

It should be noted that the embodiments of the present disclosure do not limit the deep learning. For example, the deep learning includes: a convolutional neural network, a recurrent neural network, or generative adversarial network.

It should be noted that in the embodiments of the present disclosure, the guidance image is a CBCT guidance image or a 4D-CBCT guidance image, and different target images will be obtained by different guidance images. In a case where the guidance image is the CBCT guidance image, the target image may be an intensity projection image of a 4D-CT simulation image (for example, in an implementation A). In a case where the guidance image is the 4D-CBCT guidance image, the target image may be a 4D-CT simulation image (for example, in an implementation B). Alternatively, in a case where the guidance image is the 4D-CBCT guidance image, the target image may be an intensity projection image of a 4D-CT simulation image (for example, in an implementation C).

The above three cases (i.e., the implementation A, the implementation B, and the implementation C) will be introduced below.

In the implementation A provided by the embodiments of the present disclosure, the intensity projection image of the 4D-CT simulation image may be obtained through the CBCT guidance image, which improves the image quality. The intensity projection of the 4D-CT simulation image corresponding to the CBCT guidance image may be used for image registration or target volume delineation. If the simulation image corresponding to the guidance image is used for the target volume delineation to formulate a treatment plan, since the guidance image is based on a current image of the treatment of the patient, the accuracy of the treatment plan may be improved.

For example, as shown in FIG. 3, obtaining the intensity projection image of the 4D-CT simulation image through the CBCT guidance image includes the following steps.

In S301, the CBCT guidance image is obtained.

For example, the CBCT guidance image may also be obtained through the image acquisition components of the radiation therapy system 100.

For example, obtaining the CBCT guidance image includes: obtaining an initial CBCT guidance image, and averaging the initial CBCT guidance image to obtain a CBCT intensity projection guidance image as the CBCT guidance image.

In S302, the CBCT guidance image is input into the image conversion model trained based on the deep learning to obtain the target image. The target image is the intensity projection image of the 4D-CT simulation image corresponding to the CBCT guidance image. That is to say, when the guidance image is the CBCT guidance image, the target image is the intensity projection image of the 4D-CT simulation image corresponding to the CBCT guidance image.

For example, as shown in FIG. 4, the above step S302 includes: inputting the CBCT guidance image 401 into the input layer in the trained image conversion model. Next, the input layer inputs the CBCT guidance image 401 into the hidden layer (i.e., the image processing network) to obtain the intensity projection image 402 of the 4D-CT simulation image. Then, the output layer outputs the intensity projection image 402 of the 4D-CT simulation image.

In the embodiments of the present disclosure, the intensity projection image may include a maximum intensity projection (MIP) image or an average intensity projection (AIP) image. It should be noted that the above-mentioned MIP image finally presents an image with distinct light and shade, and the highlighted part represents the maximum grayscale value in a certain direction, which is used to represent the location of blood vessel, artery or other structures. The grayscale value of the above AIP image is the average of all grayscale values of the guidance image, so the AIP image is smooth and close to the actual object shape.

In the following embodiments of the present disclosure will be illustrated by taking an example in which the intensity projection image is an AIP image. As for the case where the intensity projection image is an MIP image, reference may be made to the AIP image, and the embodiments of the present disclosure will not be described in detail here.

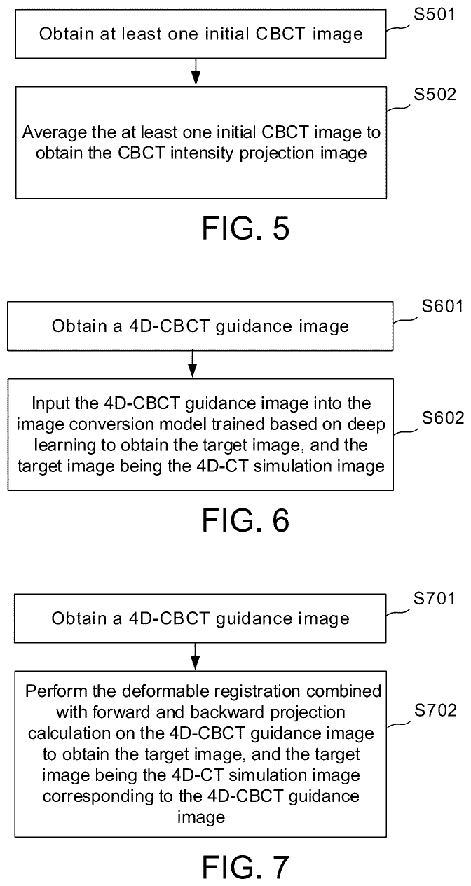

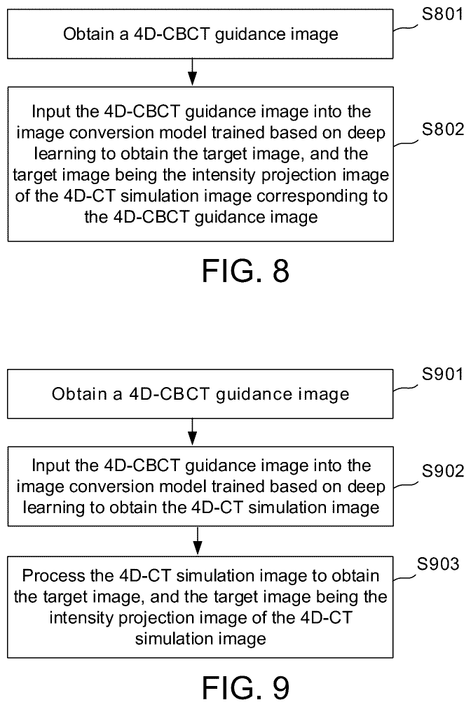

For example, in the embodiment shown in FIG. 5, the image conversion model uses CBCT images generated based on the image-guided radiation therapy system as initial images, and uses 4D-CT intensity projection images as training images. For example, the CBCT image may also be obtained through the image acquisition components of the radiation therapy system 100.

In a possible implementation, obtaining the CBCT image generated based on the image-guided radiation therapy system may include the following steps.

In S501, at least one initial CBCT image is obtained.

In S502, the at least one initial CBCT image is averaged to obtain the CBCT intensity projection image as the CBCT image. For example, the CBCT intensity projection image is an AIP image.

For example, in the embodiment shown in FIG. 5, when the guidance image is the CBCT guidance image, the processing device may train an image conversion model based on CBCT intensity projection images and 4D-CT intensity projection images to obtain the trained image conversion model.

In the embodiment provided by the present disclosure, the CBCT guidance image generated by the image-guided radiation therapy system is obtained, the image processing is performed on the CBCT guidance image, and the intensity projection image of the 4D-CT simulation image corresponding to the CBCT guidance image is generated. The intensity projection image of the 4D-CT simulation image has high definition and may be used for registration (improving registration accuracy) and target volume delineation. Since the guidance image is the current image of the patient, the intensity projection image of the 4D-CT simulation image converted from the guidance image has high definition. Therefore, the intensity projection image of the 4D-CT simulation image is used to delineate the target volume and formulate or modify the treatment plan, which may achieve precise treatment.

In some embodiments of the present disclosure, the intensity projection image of the 4D-CT simulation image includes a set of all movement positions of the tumor during the respiratory cycle. Therefore, the intensity projection image of the 4D-CT simulation image can be used for image registration to achieve precise irradiation of tumor respiratory movement.

In the implementation B provided by the embodiments of the present disclosure, the 4D-CT simulation image may be obtained by performing the image processing on the 4D-CBCT guidance image, thus improving the image quality. The 4D-CT simulation image corresponding to the 4D-CBCT guidance image may be used for three-dimensional image registration.

In some examples, performing the image processing on the 4D-CBCT guidance image to obtain the 4D-CT simulation image may be implemented by at least one of the following two implementations as shown in FIGS. 6 and 7.

In a first implementation, as shown in FIG. 6, performing the image processing on the 4D-CBCT guidance image to obtain the 4D-CT simulation image includes the following steps S601 and S602.

In S601, the 4D-CBCT guidance image is obtained.

For example, obtaining the 4D-CBCT guidance image includes obtaining a plurality of CBCT guidance images, and processing the plurality of CBCT guidance images to generate a guidance image that includes different phases within the respiratory cycle serving as the 4D-CBCT guidance image.

In S602, the 4D-CBCT guidance image is input into the image conversion model trained based on the deep learning to obtain the target image, and the target image is the 4D-CT simulation image.

In the embodiments of the present disclosure, the image conversion model uses 4D-CBCT images generated based on the image-guided radiation therapy system as initial images, and uses 4D-CT images as training images. That is to say, in a case where the guidance image is the 4D-CBCT guidance image, the processing device may train an image conversion model based on the generated 4D-CBCT images and 4D-CT images to obtain the trained image conversion model.

In a second implementation, as shown in FIG. 7, performing the image processing on the 4D-CBCT guidance image to obtain the 4D-CT simulation image may include the following steps S701 and S702.

In S701, the 4D-CBCT guidance image is obtained.

For example, obtaining the 4D-CBCT guidance image includes obtaining a plurality of CBCT guidance images, processing the plurality of CBCT guidance images to generate a guidance image that includes different phases within the respiratory cycle serving as the 4D-CBCT guidance image.

In S702, the deformable registration combined with forward and backward projection calculation is performed on the 4D-CBCT guidance image to obtain the target image. The target image is the 4D-CT simulation image corresponding to the 4D-CBCT guidance image.

The process of performing, by the processing device, the deformable registration combined with forward and backward projection calculation on the 4D-CBCT guidance image will be introduced below.

In a possible implementation, performing the deformable registration combined with forward and backward projection calculation on the 4D-CBCT guidance image includes as follows.

A planned image is obtained. For example, the planned image is a planned CT image.

A CBCT image of each phase of the 4D-CBCT guidance image is obtained.

For the CBCT image of each phase, the following steps S1 to S4 of iteration are performed.

In S1, a single-phase planned CT deformation image is subjected to forward projection to obtain a planned CT projection, and a first image is reconstructed by subtracting a projection of the CBCT image from the planned CT projection. The single-phase planned CT deformation image of the first iteration is the planned image (e.g., the planned CT image).

In S2, a second image is obtained by subtracting the reconstructed first image from the single-phase planned CT deformation image.

In S3, a current deformation field is obtained by performing the deformable registration on the second image and the planned CT image.

In S4, whether the current deformation field meets the deformation requirements is determined. If the current deformation field does not meet the deformation requirements, a current planned CT deformation image is obtained based on the current deformation field, and the current planned CT deformation image is used as a single-phase planned CT deformation image of a next iteration, and the iteration is ended until the current deformation field meets the required requirements. If the current deformation field meets the deformation requirements, the iteration is ended.

A planned CT deformation image obtained according to a deformation field that is obtained in the last iteration is used as the CT simulation image.

It can be understood that when the first iteration is performed, the single-phase planned CT deformation image is the planned CT, and the planned CT deformation image is obtained through the above steps S1 to S4. Then, the planned CT deformation image generated in the first iteration is used to perform the above steps S1 to S4 again, and so on. The planned CT deformation image obtained in the current iteration is used as the single-phase planned CT deformation image of the next iteration to perform the above steps S1-S4 again, and the iteration is ended until the deformation field meets the deformation requirements. The final output deformation image of the planned CT is the CT simulation image of the CBCT guidance image.

The 4D-CT simulation image corresponding to the 4D-CBCT guidance image is generated according to the obtained CT simulation images of a plurality of phases.

In the implementation B provided by the embodiments of the present disclosure, the 4D-CBCT guidance image is used to obtain the 4D-CT simulation image corresponding to the 4D-CBCT guidance image through different manners. The 4D-CT simulation image has high definition, and the 4D-CT simulation image may be used for image registration, thus improving registration accuracy.

In the implementation C provided by the embodiments of the present disclosure, the intensity projection image of the 4D-CT simulation image may be obtained by performing the image processing on the 4D-CBCT guidance image, which improves the image quality, and the intensity projection image of the 4D-CT simulation image corresponding to the 4D-CBCT guidance image may be used for registration or target volume delineation.

For example, performing the image processing on the 4D-CBCT guidance image to obtain the intensity projection image of the 4D-CT simulation image may be implemented through any one of the following four implementations as shown in FIGS. 8 to 11.

In a first implementation, as shown in FIG. 8, performing the image processing on the 4D-CBCT guidance image to obtain the intensity projection image of the 4D-CT simulation image includes steps S801 and S802.

In S801, the 4D-CBCT guidance image is obtained.

For example, obtaining the 4D-CBCT guidance image includes obtaining a plurality of CBCT guidance images, and processing the plurality of CBCT guidance images to generate a guidance image that includes different phases within the respiratory cycle serving as the 4D-CBCT guidance image.

In S802, the 4D-CBCT guidance image is input into the image conversion model trained based on the deep learning to obtain the target image. The target image is the intensity projection image of the 4D-CT simulation image corresponding to the 4D-CBCT guidance image.

In the embodiments of the present disclosure, the image conversion model uses 4D-CBCT images generated based on the image-guided radiation therapy system as the initial images, and uses 4D-CT intensity projection images as the training images. That is to say, the processing device may train an image conversion model based on the generated 4D-CBCT images and the 4D-CT intensity projection images, so as to obtain the trained image conversion model. For example, generating the 4D-CBCT image includes: obtaining at least one CBCT image; and processing the at least one CBCT image to generate the 4D-CBCT image.

In a second implementation, as shown in FIG. 9, performing the image processing on the 4D-CBCT guidance image to obtain the intensity projection image of the 4D-CT simulation image may include steps S901 to S903.

In S901, the 4D-CBCT guidance image is obtained.

For example, obtaining the 4D-CBCT guidance image includes obtaining a plurality of CBCT guidance images, and processing the plurality of CBCT guidance images to generate a guidance image that includes different phases within the respiratory cycle serving as the 4D-CBCT guidance image.

In S902, the 4D-CBCT guidance image is input into the image conversion model trained based on the deep learning to obtain the 4D-CT simulation image.

In S903, the 4D-CT simulation image is processed to obtain the target image, and the target image is the intensity projection image of the 4D-CT simulation image.

For example, the processing device may first obtain the 4D-CT simulation image through the model, and then perform the average processing on the 4D-CT simulation image to obtain the intensity projection image of the 4D-CT simulation image.

In a third implementation, as shown in FIG. 10, performing the image processing on the 4D-CBCT guidance image to obtain the intensity projection image of the 4D-CT simulation image may include steps S1001 and S1002.

In S1001, the 4D-CBCT guidance image is obtained.

For example, obtaining the 4D-CBCT guidance image includes obtaining a plurality of CBCT guidance images, and processing the plurality of CBCT guidance images to generate a guidance image that includes different phases within the respiratory cycle serving as the 4D-CBCT guidance image.

In S1002, the deformable registration combined with forward and backward projection calculation is performed on the 4D-CBCT guidance image to obtain the target image. The target image is the intensity projection image of the 4D-CT simulation image corresponding to the 4D-CBCT guidance image.

In a fourth implementation, as shown in FIG. 11, performing the image processing on the 4D-CBCT guidance image to obtain the intensity projection image of the 4D-CT simulation image includes steps S1101 to S1103.

In S1101, the 4D-CBCT guidance image is obtained.

For example, obtaining the 4D-CBCT guidance image includes obtaining a plurality of CBCT guidance images, and processing the plurality of CBCT guidance images to generate a guidance image that includes different phases within the respiratory cycle serving as the 4D-CBCT guidance image.

In S1102, the deformable registration combined with forward and backward projection calculation is performed on the 4D-CBCT guidance image to obtain the 4D-CT simulation image.

In S1103, the 4D-CT simulation image is processed to obtain the target image. The target image is the intensity projection image of the 4D-CT simulation image. That is to say, the processing device may first obtain the 4D-CT simulation image through registration and projection calculation, and then average the 4D-CT simulation image to obtain the intensity projection image of the 4D-CT simulation image. As for the specific processing manner, reference may be made to the above embodiments, and details will not be repeated here.

In the image processing method provided in the embodiments of the present disclosure, the 4D-CBCT guidance image may be converted into the intensity projection image of the 4D-CT simulation image through the image conversion model or the deformable registration combined with forward and backward projection calculation. Alternatively, the 4D-CBCT guidance image may be converted into the corresponding 4D-CT simulation image through the image conversion model or the deformable registration combined with forward and backward projection calculation, and then the 4D-CT simulation image is processed to obtain the intensity projection image of the 4D-CT simulation image. The intensity projection image of this 4D-CT simulation image has high definition and can be used for registration (improving registration accuracy) and target volume delineation. The guidance image is the current image of the patient, and the intensity projection image of the 4D-CT simulation image converted from the guidance image has high definition, therefore, the intensity projection image of the 4D-CT simulation image is used to delineate the target volume and formulate or modify the treatment plan, and the accurate treatment is realized.

In the embodiments of the present disclosure, the intensity projection image of the 4D-CT simulation image includes the set of all movement positions of the tumor during the respiratory cycle. Therefore, the intensity projection image of the 4D-CT simulation image may be used for image registration to realize the precise irradiation of the tumor respiratory movement.

In some embodiments, after the target image is obtained, the processing device may display the target image.

For example, the processing device may display the AIP image of the 4D-CT simulation image or the 4D-CT simulation image. It can be understood that the target image is displayed, and the target image is an electronic CT simulation image corresponding to the guidance image, so that a clear image may be displayed to the user.

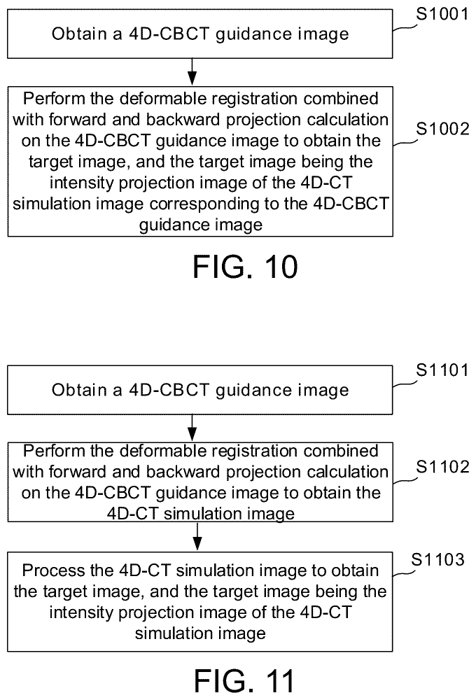

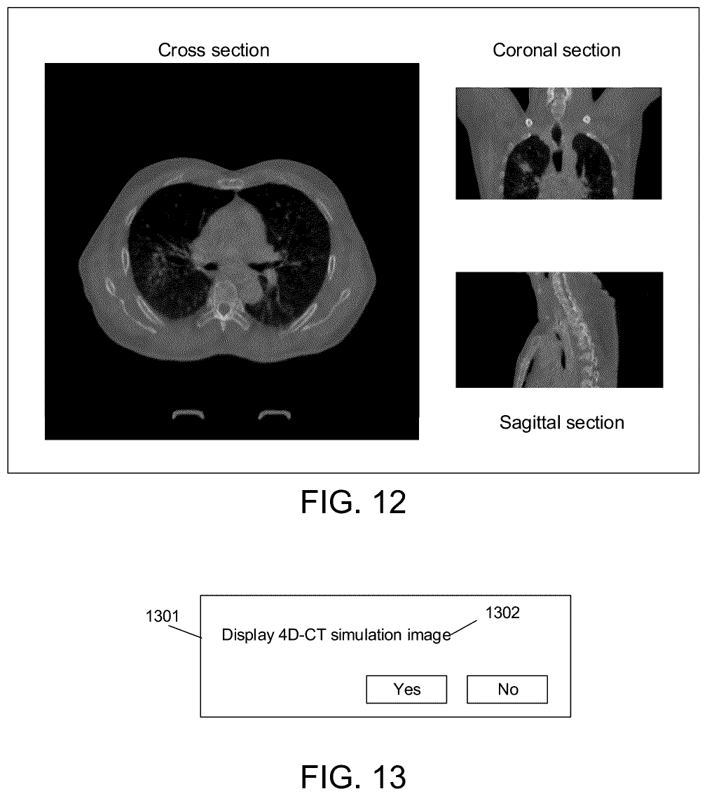

For example, as shown in FIG. 12, displaying the AIP image of the 4D-CT simulation image may include displaying a cross section view, a coronal section view, and a sagittal section view. For example, the positions of the cross section view, the coronal section view, and the sagittal section view may be adjusted according to actual needs. The embodiments of the present disclosure do not limit the size and position of each view, and the situations shown in the drawings are taken as examples for illustration. For example, three different views may be arranged in parallel.

In a possible implementation, before displaying the target image, the processing device may display first information, and the first information is used to instruct the display of the target image. Then, the processing device may display the target image in response to an operation on the first information.

For example, as shown in FIG. 13, a first interface 1301 displayed on a screen of the processing device may include prompt information 1302, and the prompt information 1302 may include a prompt box. For example, the prompt box includes “Display 4D-CT simulation image”. Optionally, the first interface 1301 displayed on the screen of the processing device may further include selection information, and the selection information may include a selection button. For example, the selection button includes “Yes” and “No”.

The first information may include the prompt information, and may further include the selection information. For example, the operation on the first information may be to click the prompt box of the prompt information, or to click the selection button of the select information.

It can be understood that by displaying the first information, it may be possible to increase the selectivity of the user, and in turn improve the use experience. The embodiments of the present disclosure do not limit the displayed prompt content of the first information. For example, it may be “Display the intensity projection image of the 4D-CT simulation image”, or other literal expressions.

For example, when the target image is the 4D-CT simulation image, displaying, by the processing device, the target image may include static display or dynamic display.

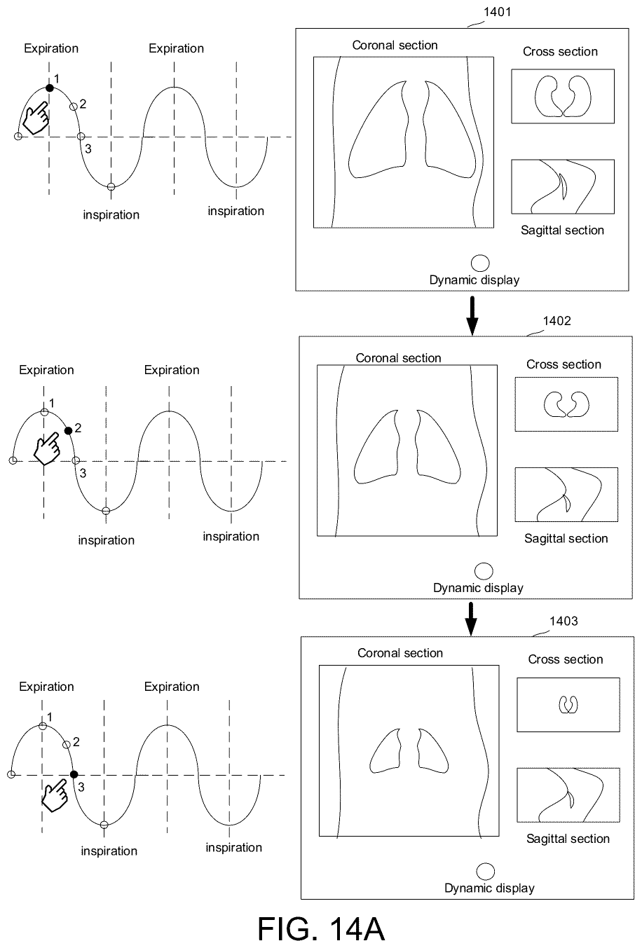

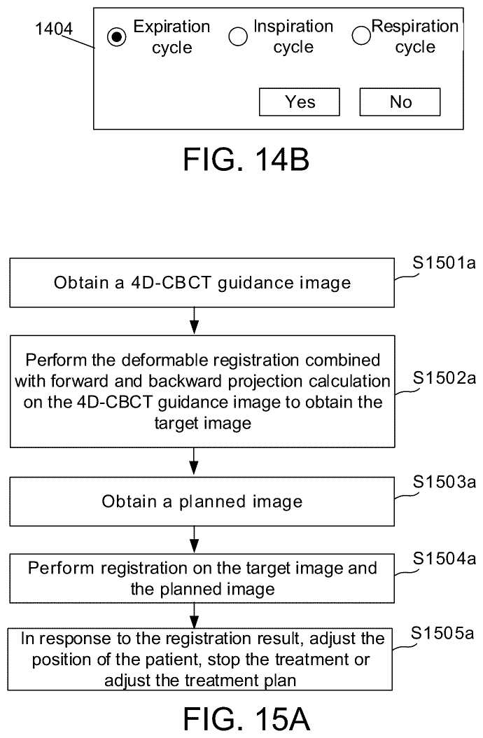

The static display refers to displaying 4D-CT simulation images corresponding to phases in response to different phases. That is, according to the phase selected by the user, the 4D-CT simulation image of the phase is displayed. For example, as shown in FIG. 14A, if the user selects phase 1, the displayed image 1401 includes three views corresponding to phase 1. If the user selects phase 2, the displayed image 1402 includes three views corresponding to phase 2. If the user selects phase 3, the displayed image 1403 includes three views corresponding to phase 3. For example, the above displayed three views are the coronal section view, the sagittal section view and the cross section view. The positions of the three views may be adjusted according to actual needs, which are not limited here.

The dynamic display refers to the dynamic display of the 4D-CT simulation image corresponding to the phase. For example, the phase and the 4D-CT simulation image corresponding to the phase may be dynamically displayed. The static display or dynamic display may be determined by the user. When the user determines the dynamic display, after receiving the dynamic display treatment, the phase and the 4D-CT simulation image of the phase will be displayed simultaneously and dynamically, e.g., will be displayed in tag image file format (TIFF). For example, as shown in FIG. 14A, the user may select the dynamic display on the interface. When the user selects the dynamic display on the interface, there is no need to select a phase, and the processing device may automatically display the phase and the views of the phase at the same time.

For example, the processing device may display the 4D-CT simulation image of the respiratory cycle (i.e., the image of the expiratory cycle and/or the image of the inspiratory cycle). Alternatively, the processing device may display an image of a first second of the expiratory cycle and/or an image of a second second of the inspiratory cycle.

Optionally, before displaying the 4D-CT simulation image of the respiratory cycle, the processing device may display second information and/or third information. The second information is used to instruct the display of a 4D-CT simulation image of at least one respiratory cycle, and the third information is used to instruct the display of 4D-CT simulation images of different phases of the respiratory cycle. The processing device displays the target image in response to an operation on the second information and/or the third information.

For example, as shown in FIG. 14B, a second interface 1404 displayed on the screen of the processing device may include option information, and the option information may include a plurality of options, such as an “Inspiratory cycle” option, an “Expiratory cycle” option, and a “Respiratory cycle” option. Moreover, the second interface 1404 displayed on the screen of the processing device may further include selection information, and the selection information may include a selection button. For example, the selection button includes “Yes” and “No”. The processing device may display one or more images in response to an operation (e.g., the clicking of the user) on the selection button. For example, when the “Inspiratory cycle” option is operated, the image of the inspiratory cycle will be displayed.

In some examples, the second information may include the option information, and may further include the selection information. The third information may include the option information, and may further include the selection information.

It can be understood that when the target image is the 4D-CT simulation image, a 4D-CT simulation image of at least one respiratory cycle may be displayed; and/or 4D-CT simulation images of different phases of the respiratory cycle may be displayed. In this way, the diversity of displayed images is enriched, which in turn may increase the basis for judgment and improve the accuracy of the judgment of the user.

In some embodiments of the present disclosure, after obtaining the target image, the registration may be performed on the target image and the image used to formulate the treatment plan, thereby improving the registration accuracy and improving the treatment accuracy. The process of the registration will be described below by taking an example in which the target image is the 4D-CT simulation image. Alternatively, the process of the registration will also be described below by taking an example in which the target image is the intensity projection image of the 4D-CT simulation image. It should be noted that, the process of the registration is described by considering the intensity projection image of the 4D-CT simulation image generated by the method shown in FIG. 7 and the intensity projection image of the 4D-CT simulation image generated by the method shown in FIG. 8 as an example, and as for the implementations of other relevant embodiments, reference may be made to the relevant description, and details will not be provided here.

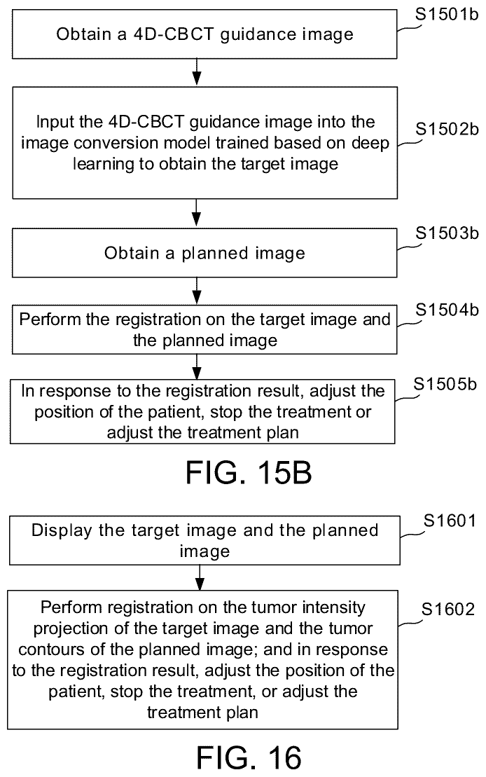

For example, as shown in FIG. 15A, after obtaining the target image, performing the registration on the target image and the image used to formulate the treatment plan may include the following steps.

In S1501a, the 4D-CBCT guidance image is obtained. As for the detailed description, reference may be made to the relevant descriptions in the above-mentioned embodiments of the present disclosure, e.g., the description of step S701, which will not be repeated here.

In S1502a, the deformable registration combined with forward and backward projection calculation is performed on the 4D-CBCT guidance image to obtain the target image (the 4D-CT simulation image). As for the detailed description, reference may be made to the relevant descriptions in the above-mentioned embodiments of the present disclosure, e.g., the description of step S702, which will not be repeated here.

In S1503a, the planned image is obtained. The planned image is a 4D-CT intensity projection image used to formulate the treatment plan.

In S1504a, the registration is performed on the target image and the planned image. As for the process of the registration, reference may be made to the relevant descriptions in the above-mentioned embodiments of the present disclosure, and details will not be repeated here.

In S1505a, in response to the registration result, the position of the patient is adjusted, the treatment is stopped or the treatment plan is adjusted.

In a possible design, the treatment plan may be used to guide radiation delivery by the radiation therapy device. The radiation therapy device includes: a radiation source for emitting a treatment beam, a frame for carrying the radiation source, and a beam collimator for controlling the shape of the beam. The treatment plan is used to guide the irradiation position of the radiation source, the duration of irradiation, the movement trajectory of the radiation source, the size/shape/position of the beam collimator, etc. Due to the guidance of the treatment plan, the radiation therapy device delivers the dose distribution specified in the treatment plan to the target volume according to the irradiation position and the movement trajectory.

For example, as shown in FIG. 15B, after obtaining the target image, performing the registration on the target image and the image used to formulate the treatment plan includes the following steps.

In S1501b, the 4D-CBCT guidance image is obtained. As for the detailed description, reference may be made to the relevant descriptions in the above-mentioned embodiments of the present disclosure, which will not be repeated here.

In S1502b, the 4D-CBCT guidance image is input into the image conversion model trained based on the deep learning to obtain the target image (the intensity projection image of the 4D-CT simulation image). As for the detailed description, reference may be made to the relevant descriptions in the above-mentioned embodiments of the present disclosure, which will not be repeated here.

In S1503b, the planned image is obtained. The planned image is a 4D-CT intensity projection image used to formulate the treatment plan.

In S1504b, the registration is performed on the target image and the planned image. As for details of the registration process, reference may be made to the relevant descriptions in the above-mentioned embodiments of the present disclosure, which will not be repeated here.

In S1505b, in response to the registration result, the position of the patient is adjusted, the treatment is stopped or the treatment plan is adjusted. For example, as for S1505b, reference may be made to the above description of S1505a, which will not be repeated here.

It can be understood that after the planned image is obtained, the registration is performed on the target image and the planned image. The guidance image is the current image of the patient, the target image is generated according to the guidance image, and the target image has high definition and good imaging effect. Therefore, a more accurate registration result may be obtained. As a result, the position of the patient may be adjusted accurately, and accordingly, the treatment may be stopped or the treatment plan may be adjusted.

In a possible implementation, as shown in FIG. 16, performing the registration on the target image and the planned image includes the following steps.

In S1601, the target image and the planned image are displayed. For example, the target image is the intensity projection image of the 4D-CT simulation image, the target image includes a tumor intensity projection, and the planned image includes tumor contours.

In S1602, the registration is performed on the tumor intensity projection of the target image and the tumor contours of the planned image; and in response to the registration result, the position of the patient is adjusted, the treatment is stopped, or the treatment plan is adjusted.

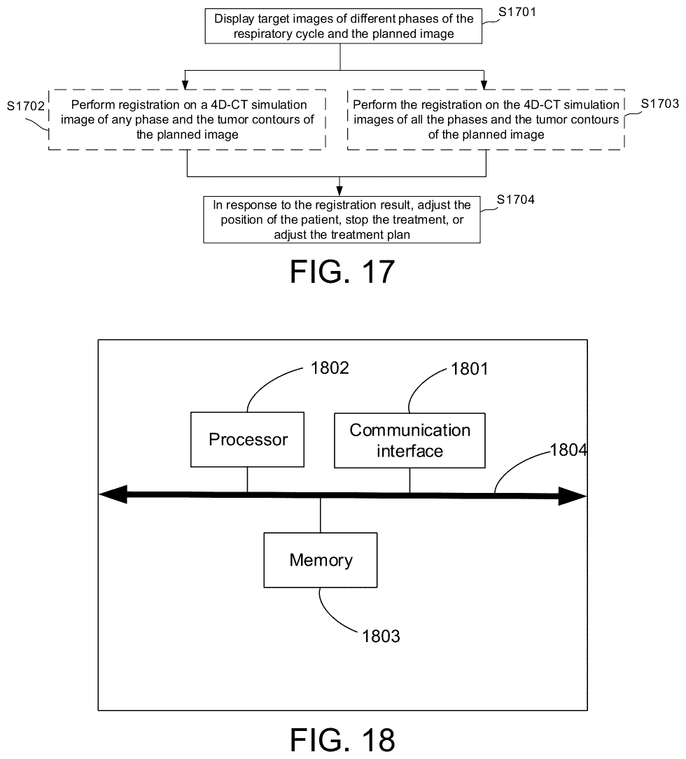

In another possible implementation, as shown in FIG. 17, performing the registration on the target image and the planned image includes the following steps.

In S1701, target images of different phases of the respiratory cycle and the planned image are displayed. The target images are 4D-CT simulation images.

In S1702, the registration is performed on a 4D-CT simulation image of any phase and the tumor contours of the planned image.

Alternatively, in S1703, the registration is performed on the 4D-CT simulation images of all the phases and the tumor contours of the planned image.

That is, after executing S1701, S1702 and S1704 may be performed. Alternatively, after executing S1701, S1703 and S1704 may be performed.

In S1704, in response to the registration result, the position of the patient is adjusted, the treatment is stopped, or the treatment plan is adjusted.

For example, the 4D-CT simulation image includes images of a plurality of phases; and the processing device may perform the registration on any one of the images of the plurality of phases and the tumor contours of the planned image, and perform a corresponding task according to the registration result.

Alternatively, the processing device may perform the registration on the 4D-CT simulation image of each phase and the tumor contours of the planned image, and in response to the registration result, adjust the patient position, stop the treatment, or adjust the treatment plan.

For example, the 4D-CT simulation image includes images of a plurality of phases; and the processing device may perform the registration on each image of the images of the plurality of phases and the tumor contours of the planned image to obtain a plurality of registration results, and perform corresponding tasks according to the plurality of registration results.

In a possible design, in a case where a side of the intensity projection of the target image deviates from the tumor contours of the planned image, the position of the patient is adjusted. It should be noted that the side of the intensity projection of the target image may be any side of the target image (such as the left side, the right side, the upper side, or the lower side).

It can be understood that if only one side of the intensity projection of the target image deviates from the tumor contours of the planned image, it means that the intensity projection of the target image is close to a position in the planned image. In this way, the treatment plan may be performed only by adjusting the position of the patient accordingly.

In another possible design, in a case where two opposite sides of the intensity projection of the target image deviate from the tumor contours of the planned image, the treatment is stopped or the treatment plan is adjusted. For example, the two sides include the left side and the right side, or the upper side and the lower side. It can be understood that if the two opposite sides of the intensity projection of the target image deviate from the tumor contours of the planned image, it means that the intensity projection of the target image deviates greatly from the position in the planned image, and the registration effect cannot be achieved by adjusting the position of the patient. Therefore, the treatment plan may be adjusted to improve the treatment accuracy. Or, the treatment plan is stopped to avoid causing additional harm.

It can be understood that, in order to implement the functions of the above method, the image processing device or electronic device (or server) includes corresponding hardware structures and/or software modules that perform the functions. Those skilled in the art may easily realize that the embodiments of the present disclosure may be implemented in hardware or in a combination of hardware and computer software with reference to steps of the image processing method of the examples described in the embodiments disclosed herein. Whether a certain function is performed by hardware or computer software driving hardware depends on specific application and design constraints of the technical solution. Professionals may implement the described functions by using different methods on various specific applications, but such implementations should not be considered beyond the scope of the present disclosure.

The embodiments of the present disclosure further provide the image processing device. The image processing device may be a computing device, or a central processing unit (CPU) in the above-mentioned computer, or a processor in the above-mentioned computer for processing images, or a client in the above-mentioned computer for processing images.

The embodiments of the present disclosure may divide the image processing device into functional modules or functional units according to the above method examples. For example, each functional modules or functional unit may be divided corresponding to each function, or two or more functions may be integrated into in a processor. The integrated module may be implemented in a form of hardware, in a form of software functional module, or in a form of functional unit. The division of modules or units in the embodiments of the present disclosure is schematic, which is merely a logical function division, and there may be other division manners in actual implementation.

FIG. 18 is a schematic structural diagram of the image processing device according to some embodiments. The image processing device may include a processor 1802, and the processor 1802 is configured to execute computer program codes (or instructions) to implement the image processing method in any of the above embodiments.

For example, the processor 1802 may be a CPU, a microprocessor, an application specific integrated circuit (ASIC), or one or more integrated circuits for controlling execution of computer program codes.

As shown in FIG. 18, the image processing device may further include a memory 1803. The memory 1803 is used to store the above computer program codes executable by the processor 1802.

The memory 1803 may be a read-only memory (ROM) or other type of static storage device that can store static information and instructions, or a random access memory (RAM) or other type of dynamic storage device that can store information and instructions, or may also be an electrically erasable programmable read-only memory (EEPROM), a compact disk read-only memory (CD-ROM) or other optical disk storage (including a compressed optical disk, a laser disk, a digital versatile disk, a Blu-ray disk, etc.), a disk storage media or other magnetic storage devices, or any other medium that can be used to carry or store desired program codes in the form of instructions or data structures and can be accessed by a computer; but it is not limited thereto. The memory 1803 may be independently arranged, and is connected to the processor 1802 through a bus 1804. The memory 1803 may also be integrated with the processor 1802.

As shown in FIG. 18, the image processing device may further include a communication interface 1801; and the communication interface 1801, the processor 1802, and the memory 1803 may be coupled to each other, for example, through the bus 1804. The communication interface 1801 is used for information interaction with other devices, for example, for supporting information interaction between the image processing device and other devices.

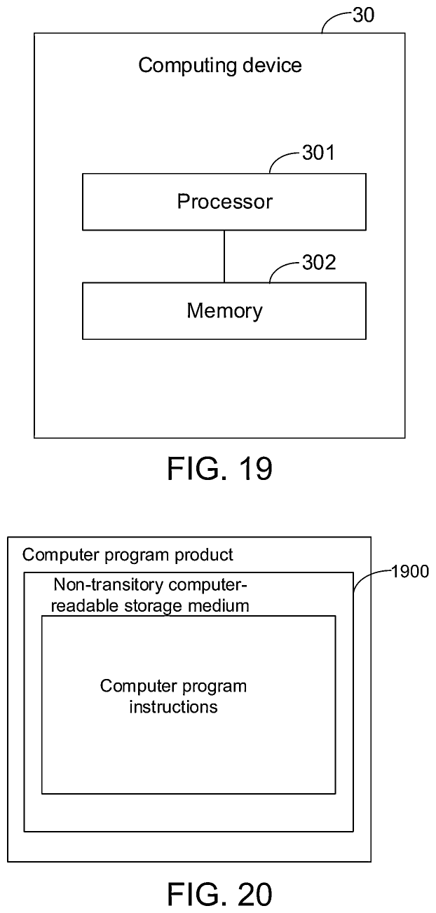

Some embodiments of the present disclosure further provide a computing device. As shown in FIG. 19, the computing device 30 includes a processor 301 and a memory 302. The memory 302 is configured to store computer program instructions executable on the processor 301. The processor 301 is configured to execute the computer program instructions to cause the computing device 30 to perform one or more steps of the image processing method in the above embodiments.

In some examples, the computing device 30 may be a computer, a laptop computer, a tablet computer, or a cloud server. The computing device 30 may further include other components, such as an input/output component, a network access component, and a bus. The processor 301 may be a CPU, a microprocessor, a general-purpose processor, a digital signal processor (DSP), an ASIC, or other programmable logic device (e.g., a field programmable gate array (FPGA)), a discrete gate or transistor logic device, a discrete hardware component, etc. The memory 302 may include a high-speed random access memory, and may also include a non-volatile memory, such as a hard disk, a memory, a plug-in hard disk, a smart media card (SMC), a secure digital (SD) card, a flash card, at least one disk memory, or a flash memory.

Beneficial effects of the above computing device are the same as the beneficial effects of the image processing method described in some of the above embodiments, which will not be repeated here.

Some embodiments of the present disclosure provide a computer-readable storage medium, the computer-readable storage medium has stored instructions that, when run on a computer, cause the computer to perform the image processing method as described in the above embodiments. The computer-readable storage medium may be a non-transitory computer-readable storage medium. For example, the non-transitory computer-readable storage medium may be a ROM, a RAM, a CD-ROM, a magnetic tape, a floppy disk, an optical data storage device, etc.

FIG. 20 shows a partial schematic diagram of the computer program product provided in the embodiments of the present disclosure. The computer program product includes a computer program for executing a computer process on a computing device.

In an embodiment, a computer program product includes a non-transitory computer-readable storage medium 1900. The non-transitory computer-readable storage medium 1900 stores computer program instructions that, when run on one or more processors, cause the one or more processors to perform one or more steps of the image processing method in any one of the above embodiments.

In some examples, the non-transitory computer-readable storage medium 1900 includes, but is not limited to, a hard driver, a compact disk (CD), a digital video disk (DVD), a digital tape, a memory, a read/write (R/W) CD, a R/W DVD, a ROM or a RAM.

The foregoing descriptions are merely specific implementation manners of the present disclosure, but the protection scope of the present disclosure is not limited thereto. Any changes or replacements that a person skilled in the art could conceive of within the technical scope of the present disclosure shall be included in the protection scope of the present disclosure. Therefore, the protection scope of the present disclosure should be determined by the protection scope of the claims.

What is claimed is:

1. An image processing method, executed by a computing device, comprising:
obtaining a guidance image, the guidance image being an image generated based on an image-guided radiation therapy system; and
performing image processing on the guidance image to generate a target image, the target image being a computed tomography (CT) simulation image corresponding to the guidance image.

2. The image processing method according to claim 1, wherein performing the image processing on the guidance image includes:
inputting the guidance image into an image conversion model trained based on deep learning; or
performing deformable registration combined with forward and backward projection calculation on the guidance image.

3. The image processing method according to claim 2, wherein the guidance image is a cone-beam computed tomography (CBCT) guidance image;
performing the image processing on the guidance image includes:
inputting the CBCT guidance image into the image conversion model trained based on deep learning to obtain the target image, wherein the target image is an intensity projection image of a four-dimensional-CT (4D-CT) simulation image corresponding to the CBCT guidance image.

4. The image processing method according to claim 3, wherein the image conversion model uses CBCT images generated based on the image-guided radiation therapy system as initial images, and uses 4D-CT intensity projection images as training images;
wherein a CBCT image generated based on the image-guided radiation therapy system is a CBCT intensity projection image obtained by averaging at least one obtained initial CBCT image.

5. The image processing method according to claim 2, wherein the guidance image is a four-dimensional-CBCT (4D-CBCT) guidance image;
performing the image processing on the guidance image includes:
inputting the 4D-CBCT guidance image into the image conversion model trained based on deep learning or performing the deformable registration combined with forward and backward projection calculation on the 4D-CBCT guidance image, so as to obtain the target image; wherein the target image is an intensity projection image of a 4D-CT simulation image corresponding to the 4D-CBCT guidance image.

6. The image processing method according to claim 5, wherein the image conversion model uses 4D-CBCT images generated based on the image-guided radiation therapy system as initial images, and uses 4D-CT intensity projection images as training images;
wherein a 4D-CBCT image generated based on the image-guided radiation therapy system is a 4D-CBCT image generated by processing at least one obtained CBCT image, and the 4D-CBCT image includes images of different phases of a respiratory cycle.

7. The image processing method according to claim 2, wherein the guidance image is a 4D-CBCT guidance image;
performing the image processing on the guidance image includes:
inputting the 4D-CBCT guidance image into the image conversion model trained based on deep learning or performing the deformable registration combined with forward and backward projection calculation on the 4D-CBCT guidance image, so as to obtain the target image; wherein the target image is a 4D-CT simulation image, and the 4D-CT simulation image corresponds to the 4D-CBCT guidance image.

8. The image processing method according to claim 2, wherein the guidance image is a 4D-CBCT guidance image;
performing the image processing on the guidance image includes:
inputting the 4D-CBCT guidance image into the image conversion model trained based on deep learning or performing the deformable registration combined with forward and backward projection calculation on the 4D-CBCT guidance image, so as to obtain a 4D-CT simulation image; and
processing the 4D-CT simulation image to obtain the target image, the target image being an intensity projection image of the 4D-CT simulation image.

9. The image processing method according to claim 8, wherein the image conversion model uses 4D-CBCT images generated based on the image-guided radiation therapy system as initial images, and uses 4D-CT images as training images;
wherein a 4D-CBCT image generated based on the image-guided radiation therapy system is a 4D-CBCT image generated by processing at least one obtained CBCT image, and the 4D-CBCT image includes images of different phases of a respiratory cycle.

10. The image processing method according to claim 8, wherein performing the deformable registration combined with forward and backward projection calculation on the 4D-CBCT guidance image includes:
obtaining a planned image, wherein the planned image is a planned CT image;
obtaining a CBCT image of each phase of the 4D-CBCT guidance image;
performing iteration on the CBCT image of each phase to generate a CT simulation image of each phase; and
obtaining CT simulation images of a plurality of phases to generate the 4D-CT simulation image corresponding to the 4D-CBCT guidance image;
wherein performing the iteration on the CBCT image of each phase includes:
performing forward projection on a single-phase planned CT deformation image to obtain a planned CT projection;
reconstructing a first image by subtracting a projection of the CBCT image from the planned CT projection, wherein a single-phase planned CT deformation image in a first iteration is the planned image;
obtaining a second image by subtracting the reconstructed first image from the single-phase planned CT deformation image;
performing deformable registration on the second image and the planned CT image to obtain a current deformation field;
determining whether the current deformation field meets deformation requirements; and
if it is determined that the current deformation field does not meet the deformation requirements, obtaining a current planned CT deformation image according to the current deformation field, and the current planned CT deformation image being used as a single-phase planned CT deformation image for a next iteration, and until it is determined that the current deformation field meets the deformation requirements, ending the iteration;
wherein a planned CT deformation image that is obtained according to a deformation field obtained in a last iteration is used as the CT simulation image.

11. The image processing method according to claim 1, further comprising: displaying the target image.

12. The image processing method according to claim 11, wherein before displaying the target image, the image processing method further comprises:
displaying first information, the first information being used to instruct display of the target image;
wherein displaying the target image includes:
displaying the target image in response to an operation on the first information.

13. The image processing method according to claim 11, wherein the target image is a 4D-CT simulation image; displaying the target image includes:
in response to different phases, displaying 4D-CT simulation images corresponding to the phases or dynamically displaying the 4D-CT simulation images corresponding to the phases.

14. The image processing method according to claim 1, further comprising:
obtaining a planned image, wherein the planned image is a 4D-CT intensity projection image used to formulate a treatment plan;
performing registration on the target image and the planned image; and
in response to a registration result, adjusting a position of a patient, stopping treatment, or adjusting the treatment plan.

15. The image processing method according to claim 14, wherein performing the registration on the target image and the planned image includes:
displaying the target image, wherein the target image is an intensity projection image of a 4D-CT simulation image, and the target image includes a tumor intensity projection;
displaying the planned image, wherein the planned image includes tumor contours; and
performing the registration on the tumor intensity projection of the target image and the tumor contours of the planned image.

16. The image processing method according to claim 14, wherein performing the registration on the target image and the planned image includes:
displaying target images of different phases of a respiratory cycle, wherein the target images are 4D-CT simulation images;
displaying the planned image, wherein the planned image includes tumor contours; and
performing the registration on a 4D-CT simulation image of any phase and the tumor contours of the planned image, or performing the registration on a 4D-CT simulation image of each phase and the tumor contours of the planned image.

17. The image processing method according to claim 14, wherein in response to a registration result, adjusting the position of the patient, stopping the treatment or adjusting the treatment plan includes:
when a side of an intensity projection of the target image deviates from tumor contours of the planned image, adjusting the position of the patient; and
when two opposite sides of the intensity projection of the target image deviate from the tumor contours of the planned image, stopping the treatment or adjusting the treatment plan.

18. The image processing method according to claim 3, wherein the intensity projection image of the 4D-CT simulation image includes a maximum intensity projection (MIP) image or an average intensity projection (AIP) image.

19. A computing device, comprising:
a processor; and
a memory coupled to the processor, wherein the memory is used to store one or more programs, and the one or more programs include computer program instructions that, when executed by the processor, cause the computing device to perform the image processing method according to claim 1.

20. A non-transitory computer-readable storage medium having stored computer program instructions that, when run on a computer, cause the computer to perform the image processing method according to claim 1.
